(19) United States
(12) Patent Application Publication
(10) Pub. No.: US 2008/0161722 A1
(43) Pub. Date: Jul. 3, 2008

(54) ASPIRATION DEVICE AND METHOD FOR CELLS AND TISSUES

(75) Inventors: Yeou-Bin Guu, Taichung City (TW); Jan-Hao Chen, Changhua County (TW); Lih-Tao Hsu, Taoyuan County (TW); Wann-Hsin Chen, Hsinchu City (TW)

Correspondence Address: QUINTERO LAW OFFICE, PC, 2210 MAIN STREET, SUITE 200, SANTA MONICA, CA, 90405, US

(73) Assignee: INDUSTRIAL TECHNOLOGY RESEARCH INSTITUTE, HSINCHU (TW)

(21) Appl. No.: 11/690,064

(22) Filed: Mar. 22, 2007

(30) Foreign Application Priority Data
Dec. 29, 2006 (TW) TW95149883

(51) Int. Cl. A61B 10/02

(52) U.S. Cl. 600/571

(57) ABSTRACT

An aspiration device for cells or tissues loaded on a container includes a tubular body, a scraper disposed on the tubular body. The scraper scrapes cells or tissues from the container, and the scraped cells or tissues are suctioned into the tubular body. The scraper includes a connecting portion connected to the tubular body, an elastic portion connected to the connecting portion, and a scraping end connected to the elastic portion. When the scraper scrapes the cells or the tissues, the elastic portion deforms elastically to enable the scraping end to abut the surface of the container, thus, the cells or tissues are scraped.

### BACKGROUND OF THE INVENTION

1. Field of the Invention

The invention relates to an aspiration device and method, and more particularly relates to an aspiration device with a scraper for scraping and aspirating cells or tissues.

2. Description of the Related Art

With advances in biotechnology, the number of operations on organisms, such as dissection and aspiration, are increased and labor intensive. The cultivation of human embryonic stem cells is described as an example. The embryonic stem cells are cultivated in a container as a colony. As the cell colony grows, the colonies should be passaged dissecting to small pieces and transfer to a new containers for further cultivation. As the cells are loaded on the wall of the container, U.S. Pat. No. 5,957,864 discloses a device such as a syringe for suctioning free cells. Such a device, however, is unable to aspirate cells adhered to the surface of the container.

### BRIEF SUMMARY OF INVENTION

The invention provides an aspiration device for cells or tissues loaded on a container. An embodiment of an aspiration device for cells or tissues loaded on a container comprises a tubular body, a scraper disposed on the tubular body. The scraper scrapes cells or tissues from the container, and the scraped cells or tissues are suctioned into the tubular body.

The scraper comprises a connecting portion connected to the tubular body, an elastic portion connected to the connecting portion, and a scraping end connected to the elastic portion. When the scraper scrapes the cells or the tissues, the elastic portion elastically deforms enabling the scraping end to abut the surface of the container, thus, the cells or the tissues are scraped from the surface.

The elastic portion is U-shaped or π-shaped with one end connected to the connecting portion and the other end connected to the scraper.

The scraper and the tubular body are preferably integrally formed.

The scraper comprises a metal, alloy, glass, ceramic or plastic material.

The invention further comprises a piston reciprocating in the tubular body to generate suction force.

The invention further comprises a pipe connected to the tubular body and a piston reciprocating in the pipe to generate suction force.

A detailed description is given in the following embodiments with reference to the accompanying drawings.

### BRIEF DESCRIPTION OF DRAWINGS

The invention can be more fully understood by reading the subsequent detailed description and examples with references made to the accompanying drawings, wherein:

FIG. 1 is a schematic view of an embodiment of an aspiration device of the invention;

FIG. 2 is an enlarged view of the tubular body and the scraper of FIG. 1;

FIG. 3 is a schematic view showing an aspiration device of the invention installed in a machine; and

FIG. 4 is a schematic view of another embodiment of an aspiration device of the invention.

### DETAILED DESCRIPTION OF INVENTION

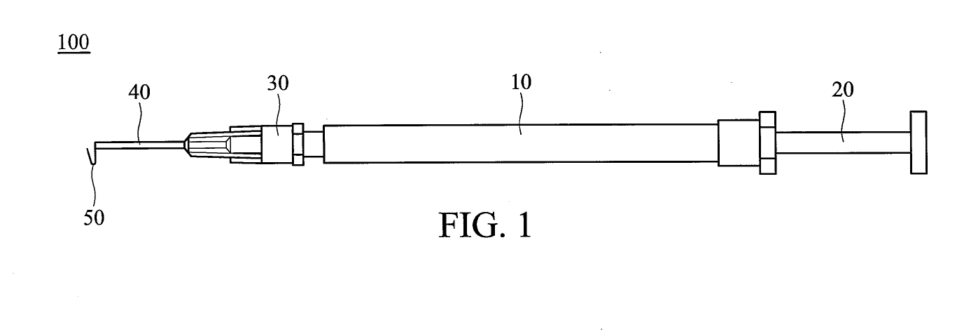

Referring to FIG. 1, an embodiment of the aspiration device 100 of the invention comprises a pipe 10, a piston 20, a connecting body 30, a tubular body 40 and a scraper 50. The piston 20 is capable of reciprocating in the pipe 10 to generate suction or discharging force, thus, objects are discharged or suctioned. The tubular body 40 is connected to the pipe 10 by the connecting body 30. The scraper 50 is connected to the outer surface of the tubular body 40.

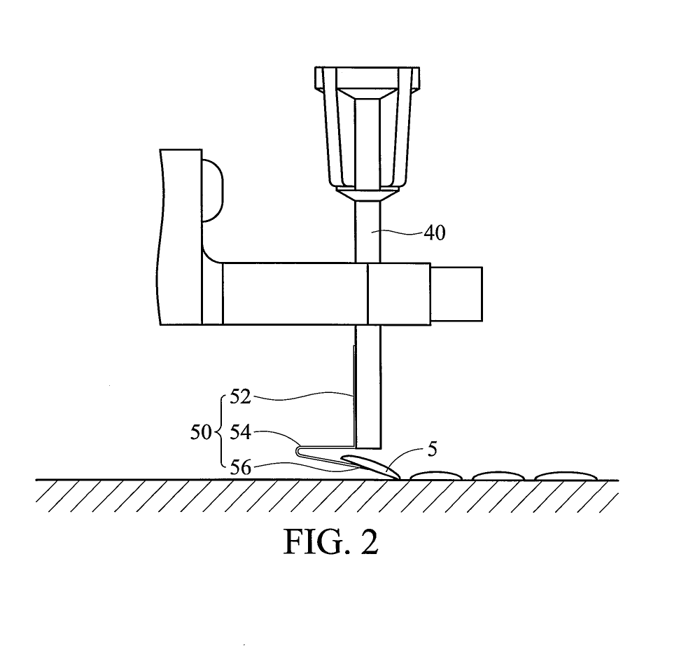

Referring to the FIG. 2, the scraper 50 comprises a connecting portion 52, an elastic portion 54 and a scraping end 56. The connecting portion 52 is linear and connected to the tubular body 40. The elastic portion 54 is U-shaped with one end connected to the connecting portion 52 and the other end connected to the scraping end 56 to scrape the cells 5 from a container.

When cells or tissues are to be scraped, the aspiration device 100 descends to enable the scraping end 56 of the scraper 50 to abut the surface of the container. As the elastic portion 54 is elastically deformable, the scraping end 56 maintains it position abutting the surface of the container, thus, cells are easily scraped from the container. As the piston 20 reciprocates in the pipe 10 to generate suction force and the tubular body 40 is connected to the pipe 10, the scraped cells are suctioned into the tubular body 40 and discharged into another container.

The tubular body 40 can also be connected to a pump or other device generating suction or discharging force to suction the scraped cells into the tubular body 40 or discharge the suctioned cells into another container.

Although the described elastic portion 54 is U-shaped, the invention is not limited to this. A π-shaped elastic portion is also applicable. The thickness of the scraper 50 is not limited, but is preferably between 0.05 mm and 0.1 mm.

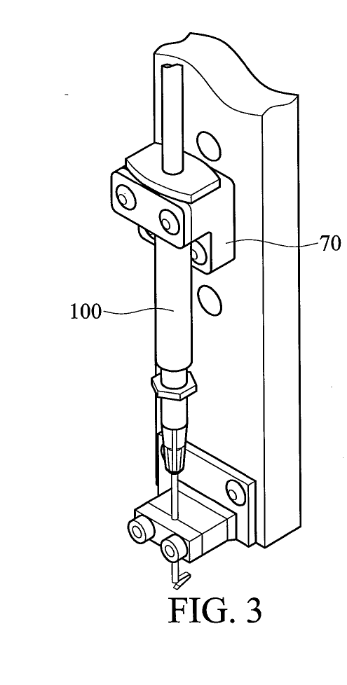

Referring to FIG. 3, the aspiration device 100 is installed on a machine 70 raising or lowering the aspiration device 100 and moving the scraper 50 to scrape cells from the container.

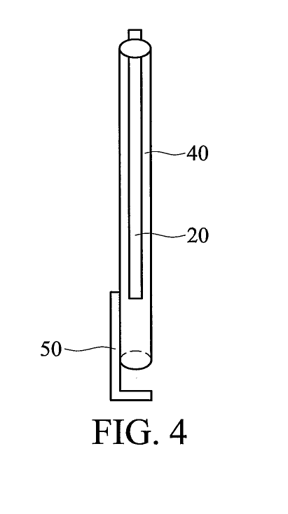

FIG. 4 depicts another embodiment of the aspiration device of the invention. In this embodiment, the piston 20 is disposed in the tubular body 40 and reciprocates therein to generate suction or discharging force. The scraper 50 is the same as in the previously described embodiment.

The scraper 50 can be welded to the tubular body 40 or integrally formed with the tubular body 40. The material of the scraper 50 can be metal, alloy, glass, ceramic or plastic.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

1. An aspiration device for cells or tissues loaded on a container, comprising
a tubular body;
a scraper disposed on the tubular body and comprising a connecting portion fixed to the tubular body, an elastic portion connected to the connecting portion, and a scraping end connected to the elastic portion, wherein the when the scraper scrapes the cells or the tissues from the container, the elastic portion deforms elastically to enable the scraping end to abut the surface of the container so as to scrape the cells or the tissues, and the scraped cells or tissues are suctioned into the tubular body.

2. (canceled)

3. The aspiration device as claimed in claim 1, wherein the elastic portion is U-shaped with one end connected to the connecting portion and the other end connected to the scraper.

4. The aspiration device as claimed in claim 1, wherein the elastic portion is II-shaped with one end connected to the connecting portion and the other end connected to the scraper.

5. The aspiration device as claimed in claim 1, wherein the scraper and the tubular body are integrally formed.

6. The aspiration device as claimed in claim 1, wherein the material of the scraper comprises metal, alloy, glass, ceramics or plastic.

7. The aspiration device as claimed in claim 1 further comprising a piston reciprocating in the tubular body to generate suction force.

8. The aspiration device as claimed in claim 1 further comprising a pipe connected to the tubular body and a piston reciprocating in the pipe to generate suction force.

9. The aspiration device as claimed in claim 1, wherein the scraper has a thickness of 0.05 mm˜0.1 mm.

10. An aspiration method for cells or tissues loaded on a container, comprising the following steps:
providing an aspiration device as claimed in claim 1;
enabling the scraper to abut the surface of the container and moving the scraper to scrape the cells or the tissues out; and
suctioning the scraped cells or tissues into the tubular body.
